(19) United States
(12) Patent Application Publication
(10) Pub. No.: US 2017/0215732 A1
(43) Pub. Date: Aug. 3, 2017

(54) ESOPHAGEAL MARKING, OCT METHOD, AND TREATMENT METHOD USING MARKS

(71) Applicant: Corning Incorporated, Corning, NY (US)

(72) Inventors: Michael Lucien Genier, Horseheads, NY (US); Michael Paul Then, Victor, NY (US)

(21) Appl. No.: 15/327,739

(22) PCT Filed: Jul. 21, 2015
(86) PCT No.: PCT/US2015/041254

§ 371 Date: Jan. 20, 2017

Related U.S. Application Data
(60) Provisional application No. 62/026,873, filed on Jul. 21, 2014.

(51) Int. Cl. A61B 5/00; A61B 90/00

(52) U.S. Cl. CPC A61B 5/0035; A61B 5/0066; A61B 5/0086; A61B 5/6853; A61B 90/37; A61B 90/39; A61B 2090/3735; A61B 2090/3979; A61B 2090/3995

(57) ABSTRACT

According to one embodiment, a balloon catheter includes a balloon located at a distal end of the balloon catheter. The balloon includes a first transferrable marking and a second transferrable marking on an exterior surface of the balloon. The first transferrable marking includes a partially IR-transmittable marking material. The second transferrable marking includes an IR-transmittable marking material. An OCT probe including the balloon catheter and a method of using the OCT probe are also disclosed.

### CROSS-REFERENCE TO RELATED APPLICATIONS

This application claims the benefit of U.S. Provisional Application Ser. No. 62/026,873 filed on Jul. 21, 2014, which is hereby incorporated by reference in its entirety.

### FIELD

The present specification generally relates to balloon catheters and, more specifically, to balloon catheters including external, transferrable markings.

### TECHNICAL BACKGROUND

A variety of medical conditions, including Barrett's Esophagus and esophageal cancer, are diagnosed, treated, and monitored using imaging techniques such as optical coherence tomography (OCT). For example, diagnosis of Barrett's Esophagus includes biopsying the esophagus based on visual inspection. The use of the OCT technique generates an OCT image that may be used to reveal underlying tissue morphologies using infrared light, enabling a physician to further investigate anomalies beneath the surface.

The OCT imaging technique uses a balloon catheter with an inner scanning optical system. The scanning optical system generates a 3-dimensional image of the scanned area of the esophagus. Once the scan is complete, the balloon catheter is removed through the endoscope. In order to return to the locations identified as diseased tissue or as anomalies to perform a biopsy or monitor a previously biopsied location, the physician conventionally relies on his or her own visual comparison of the esophagus with the OCT image. Such an approach can be difficult, as there are no direct references or registration features to match with the image within the esophagus and there may be no visual clues within the esophagus to identify the diseased tissue.

Accordingly, a need exists for alternative methods and apparatuses for correlating a location within the esophagus with a corresponding location on an image of the esophagus.

### SUMMARY

According to one embodiment, a balloon catheter includes a balloon located at a distal end of the balloon catheter. The balloon includes a first transferrable marking and a second transferrable marking on an exterior surface of the balloon. The first transferrable marking includes a partially IR-transmittable marking material. The second transferrable marking includes an IR-transmittable marking material.

According to another embodiment, an optical coherence tomography (OCT) probe includes an IR-optical imaging device for communication with an imaging system and a balloon catheter having a balloon at a distal end of the balloon catheter. The balloon includes a plurality of transferrable markings on an exterior surface of the balloon. A first portion of the plurality of transferrable markings includes a partially IR-transmittable marking material and a second portion of the plurality of transferrable markings includes an IR-transmittable marking material. The IR-optical imaging device senses the first portion of the plurality of markings and does not sense the second portion of the plurality of markings.

According to yet another embodiment, a method for performing optical coherence tomography (OCT) includes positioning an OCT imaging device adjacent to an area for investigation; transferring a plurality of markings from the OCT imaging device to the adjacent area for investigation; and transmitting an image from the optical imaging device. A first portion of the plurality of markings includes a partially IR-transmittable marking material and a second portion of the plurality of markings includes an IR-transmittable marking material. The image transmitted from the optical imaging device includes at least the first portion of the plurality of markings and a three-dimensional model of the area for investigation.

Additional features and advantages will be set forth in the detailed description which follows, and in part will be readily apparent to those skilled in the art from that description or recognized by practicing the embodiments described herein, including the detailed description which follows, the claims, as well as the appended drawings.

It is to be understood that both the foregoing general description and the following detailed description describe various embodiments and are intended to provide an overview or framework for understanding the nature and character of the claimed subject matter. The accompanying drawings are included to provide a further understanding of the various embodiments, and are incorporated into and constitute a part of this specification. The drawings illustrate the various embodiments described herein, and together with the description serve to explain the principles and operations of the claimed subject matter.

### BRIEF DESCRIPTION OF THE DRAWINGS

FIG. 1 schematically depicts an optical coherence tomography (OCT) probe in accordance with one or more embodiments described herein;

FIG. 2 schematically depicts an inflated balloon including transferrable markings in accordance with one or more embodiments described herein;

FIGS. 3A and 3B schematically depict a cross-section of a balloon including transferrable markings in accordance with one or more embodiments described herein;

FIGS. 4A-4D schematically depict an inflated balloon including transferrable markings in accordance with one or more embodiments described herein;

FIG. 5 schematically depicts transferrable markings including partially IR-transmittable marking material in accordance with one or more embodiments described herein;

FIG. 6 schematically depicts a flat view of the transferrable markings including partially IR-transmittable marking material in accordance with one or more embodiments described herein;

FIG. 7 schematically depicts a slice of an image in accordance with one or more embodiments described herein;

FIG. 8 schematically depicts a depth profile of an image in accordance with one or more embodiments described herein;

FIG. 9 schematically depicts a pre-operative cross-section of an area for investigation according to one or more embodiments described herein;

FIG. 10 schematically depicts a post-operative cross-section of the area for investigation shown in FIG. 9 according to one or more embodiments described herein;

FIG. 11 schematically depicts an inflated balloon including transferrable markings in accordance with one or more embodiments described herein;

FIG. 12 schematically depicts a pre-operative cross-section of an area for investigation according to one or more embodiments described herein;

FIG. 13 schematically depicts an inflated balloon including transferrable markings in accordance with one or more embodiments described herein;

FIG. 14 schematically depicts a cross-section of a deflated balloon in accordance with one or more embodiments described herein;

FIG. 15 is a plot of the transmittance (y-axis) of an exemplary ink as a function of wavelength (x-axis) in the visible and IR range; and

FIG. 16 is a plot of the transmittance (y-axis) of an exemplary ink as a function of wavelength (x-axis) in the far-IR range.

### DETAILED DESCRIPTION

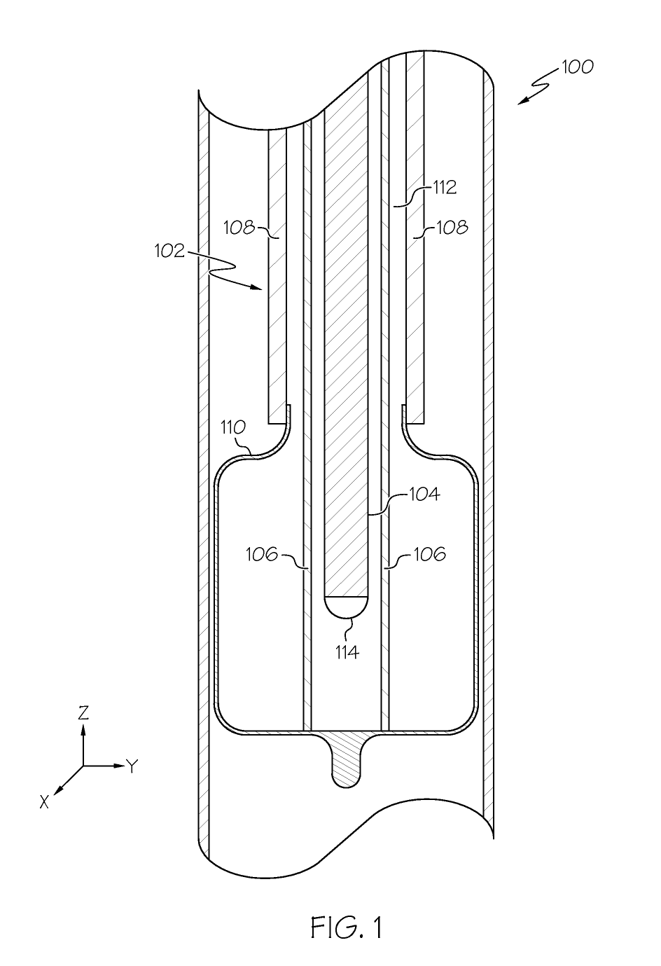

Reference will now be made in detail to various embodiments of balloon catheters having transferable markings and optical coherence tomography probes comprising the same, examples of which are illustrated in the accompanying drawings. Whenever possible, the same reference numerals will be used throughout the drawings to refer to the same or like parts. One embodiment of the components of the present disclosure is shown in FIG. 1, and is designated generally throughout by the reference numeral 100. The components generally may include an optical coherence tomography (OCT) probe including a balloon catheter and an IR-optical imaging device. The balloon catheter includes a balloon located at a distal end of the balloon catheter. The balloon includes transferrable markings on the exterior of the balloon. At least some of the markings include an IR-transmittable marking material while at least others of the markings include a partially IR-transmittable marking material. When the balloon is inflated to bring the exterior surface of the balloon into contact with an area for investigation, the transferrable markings are transferred to the area for investigation. Accordingly, when the IR-optical imaging device scans the area for investigation to generate an image, the image will include the partially IR-transmittable markings and not the IR-transmittable markings. Various embodiments of balloon catheters and OCT probes comprising the same will be described with specific reference to the appended drawings.

Referring now to FIG. 1, one embodiment of an OCT probe 100 is schematically depicted. The OCT probe 100 has a proximal end and a distal end. The OCT probe 100 generally includes a balloon catheter 102 and an IR-optical imaging device 104. The balloon catheter 102 includes an inner lumen 106 positioned within an outer lumen 108, and a balloon 110 at a distal end of the balloon catheter 102. The inner lumen 106 extends through the balloon 110. The area between the inner lumen 106 and the outer lumen 108 forms a fluid passageway 112 to transfer a fluid to and from the balloon 110 to inflate and deflate the balloon 110. In various embodiments, the fluid is a gas, but liquids may be used in some embodiments. The balloon 110 may be made of nylon, polyurethane, PET (e.g., Mylar®), or another suitable material. In various embodiments, the material is selected such that the balloon 110 is transparent to the IR-optical imaging device 104 enabling the IR-optical imaging device 104 can capture images through the balloon 110.

The OCT probe 100, is connected at its proximal end to an interferometric sensing system (not shown), such as is described in U.S. Pat. No. 7,366,376, which is hereby incorporated by reference. The IR-optical imaging device 104 located at the distal end of the probe and is positioned within the inner lumen 106, as shown in FIG. 1. When the IR-optical imaging device 104 is positioned within the inner lumen 106, the inner lumen 106 is IR-transparent such that the IR-optical imaging device 104 is able to capture images through the inner lumen 106. In various embodiments, the inner lumen 106 is transparent to both IR and white light.

The IR-optical imaging device 104 is communicatively coupled to the interferometric sensing system is configured to capture an image through the balloon 110. The interferometric sensing system may be capable of capturing an image in 360 degrees. In one method, the IR-optical imaging device 104 may be rotated by applying rotary motion to the proximal end of the IR-optical imaging device 104, to enable the interferometric sensing system to capture a 360-degree image. In some embodiments, the IR-optical imaging device 104 may be moved longitudinally (e.g., along the z-axis) to capture 360-degree images over the entire length of the balloon 110. The IR-optical imaging device 104 and the interferometric sensing system provide cross-sectional images using imaging reflections from within adjacent tissue. In particular, the IR-optical imaging device 104 directs an optical beam at an adjacent area for investigation, and a portion of the light that is reflected from the surface and sub-surface features of the esophagus is collected. The use of an OCT technique enables clear 3D images into the depth of the tissue being investigated.

In various embodiments, the IR-optical imaging device 104 may be stationary. In such embodiments, a device 114 is used to manipulate the angular position of the optical probing beam towards the adjacent area for investigation. The device 114 in this embodiment is a DLP (Digital Light Processer) as commercially available from sources such as Texas Instruments. The device 114 actively couples the probe light emitted from the distal end of the IR-optical imaging device 104 to the adjacent area for investigation, and communicatively transmits the reflected light to the interferometric sensing system to display the image on a display device associated with the imaging system. Alternative means for manipulating probe light, such as using piezoelectric devices to manipulate the position of the imaging device toward specific locations, may be employed.

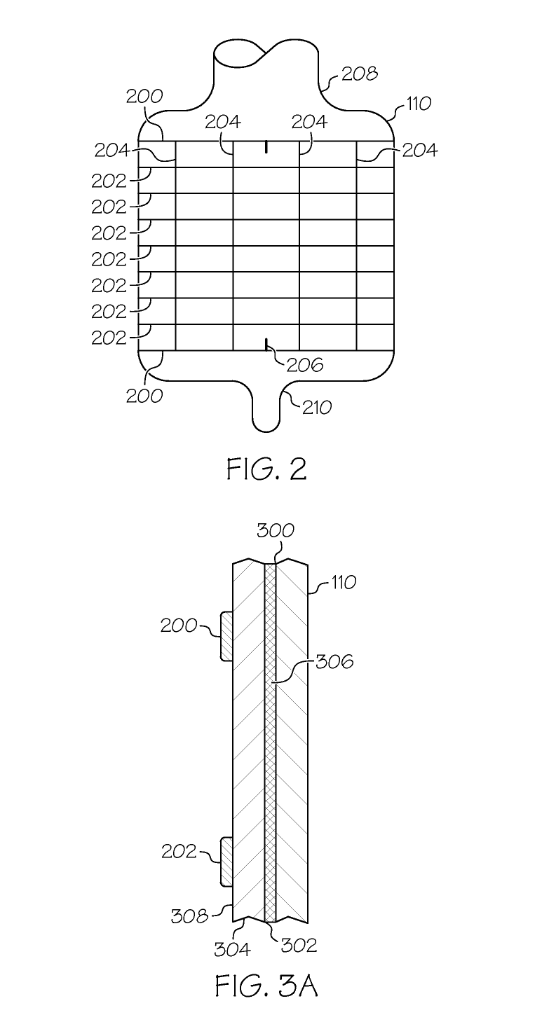

Turning now to FIG. 2, the balloon 110 includes transferrable markings on an exterior surface of the balloon. In particular, the transferrable markings include transferrable markings 200 formed from a partially IR-transmittable marking material and transferrable markings 202 formed from an IR-transmittable marking material. In the embodiment depicted in FIG. 2, the balloon 110 further includes transferrable markings 204 and 206. Although the transferrable markings 200, 202, 204, and 206 depicted in FIG. 2 form a grid on the exterior surface of the balloon, it is contemplated that in various embodiments, the markings can form other shapes, graphics, or take other forms. As shown in the embodiment depicted in FIG. 2, the transferrable markings 200 include a horizontal line located near the proximal end 208 of the balloon 110 and a horizontal line located near the distal end 210 of the balloon 110. The transferrable markings 200 include a partially IR-transmittable marking material. In other words, when the transferrable markings 200 are scanned with the IR-optical imaging device 104, the IR-optical imaging device 104 senses the transferrable markings 200.

The transferrable markings 202 depicted in FIG. 2 include a plurality of horizontal lines located between the horizontal line located near the proximal end of the balloon 110 and the horizontal line located near the distal end of the balloon 110. The transferrable markings 204 include a plurality of vertical lines that extend from the horizontal line located near the proximal end of the balloon 110 to the horizontal line located near the distal end of the balloon. Together, the transferrable markings 202 and 204 form a transferrable grid. The transferrable markings 202 and 204 are formed from an IR-transmittable marking material. In other words, when the transferrable markings 202 and 204 are scanned with the IR-optical imaging device 104, the IR-optical imaging device 104 does not sense the transferrable markings 202 and 204.

In various embodiments, such as the embodiment depicted in FIG. 2, the transferrable markings further include at least one partially IR-transmittable longitudinal marking 206. The partially IR-transmittable longitudinal marking 206 serves as an angular registration point to orient the transmitted image with respect to the cylinder of the esophagus, as will be discussed in greater detail below.

In various embodiments, both the partially IR-transmittable marking material and the IR-transmittable marking material are visible in white light. The use of marking material that is visible in white light enables a physician to visualize the transferrable markings using a white light during excision or other exploration of the area. The partially IR-transmittable marking material and the IR-transmittable marking material may be an FDA-approved dye, such as those described herein and otherwise known in the art. For example, in embodiments, the partially IR-transmittable marking material may be Adam Gates LIT IR 1151 and the IR-transmittable marking material may be Adam Gates DYE VIS606. As another example, the partially IR-transmittable marking material may be CARCO WS-860 black ink, which is FDA-approved. In various embodiments, the partially IR-transmittable marking material has a transmittance of from about 25% to about 95% transmittance in the IR spectrum. In some embodiments, the partially IR-transmittable marking material has a transmittance of greater than about 25% transmittance in the IR spectrum and/or less than about 93% transmittance in the IR spectrum.

In some embodiments, the dye is blended with an FDA-approved adhesive or sealant, such as a PEG hydrogel or a cyanoacrylate. For example, the marking material may be a combination of a dye and a PEG sealant. However, it is contemplated that a variety of sealants and/or adhesives may be used, provided that they are able to bond to wet surfaces, such as the esophagus. In various embodiments, one or more coatings are applied over the marking material to enhance various properties of the marking material. For example, a water-soluble protective layer, such as a shellac (e.g., food grade E904), may be applied over the marking material to prevent blocking of the adhesive. As another example, a time-release coating may be positioned over the marking material to prevent the marking material from being transferred before the balloon 110 is in place adjacent to a selected area of investigation.

In some embodiments, the transferrable markings 200, 202, 204, and 206 are transferred from the exterior surface of the balloon 110 to an adjacent area selected for investigation, such as a portion of a patient's esophagus, by contact. For example, the balloon 110 may be initially inserted into the esophagus in a deflated state and thereafter inflated such that the exterior surface of the balloon 110 is in contact with an adjacent area of the esophagus selected for investigation. Once the balloon 110 is inflated, the IR-optical imaging device 104 may begin scanning the adjacent area for investigation. While the IR-optical imaging device 104 is scanning the adjacent area, the transferrable markings 200, 202, 204, and 206 are released from the exterior surface of the balloon 110 and adhere to the adjacent area for investigation. In such embodiments, the marking material is selected such that it will be released from the exterior surface of the balloon 110 after being in contact with the adjacent area for from about 5 to about 10 seconds. The transferrable markings 200, 202, 204, and 206 are transferred within about 3 minutes, within about 5 minutes, within about 10 minutes, or within about 15 minutes after the balloon 110 is inflated adjacent to the area for investigation. In some embodiments, the marking material may be fully set in less than about 60 seconds in order to prevent smudging.

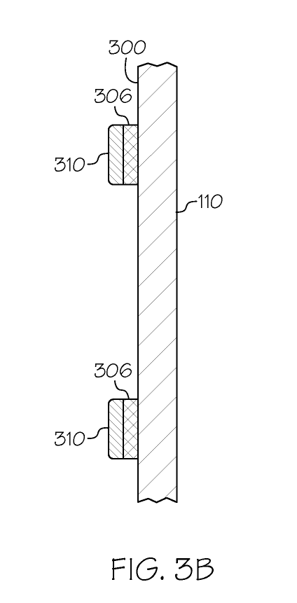

In some embodiments in which the transferrable markings are transferred by contact, the marking material may be in the form of a laminated structure on the exterior surface of the balloon 110, as shown in FIG. 3A. In FIG. 3A, the exterior surface 300 of the balloon 110 is positioned adjacent to a non-release surface 302 of a silicone release liner 304. In various embodiments, the non-release surface 302 of the silicone release liner 304 may be adhered to the exterior surface 300 of the balloon 110 through an adhesive layer 306. The silicone release liner further includes a release surface 308. The transferrable markings 200 and 202 are disposed on the release surface 308 of the silicone release liner 304. Thus, when the balloon 110 is inflated and the transferrable markings 200 and 202 contact the adjacent area for investigation, the transferrable markings 200 and 202 are released from the silicone release liner 304 and adhere to the adjacent area for investigation.

In other embodiments in which the transferrable markings 200 and 202 are transferred by contact, the balloon 110 has water soluble transferrable markings in the form of shaped pieces 310 adhesively attached to it via an adhesive layer 306, as shown in FIG. 3B. The transferrable markings 200 and 202 are made with a water soluble dye infused into a polymer compound. The water soluble polymer compounds can be, for example, casein, gum Arabica, dextran, or other similar synthetic FHA water soluble compounds. One or more dyes are added to the water soluble polymer. The dye can be chosen from a large number of FDA-approved dyes, including but not limited to the dyes commonly known as tartrazine (acid yellow 23), Allura red AC (Curry red #40), fluorescein diacetate, and FD&C Blue #2.

The polymer material and dye are heated and mixed to a uniform consistency. Some portion of the mixing can be conducted via a process called calendering. This calendaring process is well known in the art, utilizing opposing roller to form thin sheets or ribbons of the polymer compound. Rough calendering can be used to mix the base polymer and the dye uniformly, and fine calendering is used to create polymer layers or sheets of precise thicknesses known as polymer stock sheets.

The precisely calendered dye infused polymer sheet stock is then processed via a number of means, including die cutting. Die cutting is a process in which punch and hole are aligned and pressed together whilst a sheet of material is placed between the punch and hole. Once pressure is exerted, the punch, pushed a portion of the sheet material through the hole, extracting a small portion from the larger sheet of material. In various embodiments, the polymer stock sheet is die cut in to shaped pieces 310. The shaped pieces 310 can be punched into various shapes including circular, square, octagon, hexagon, star, rectangular trapezoidal, “V” shaped (or other letters or symbols) or other shapes that enable detection by surgeons and the IR-optical imaging device 104 and use as a tool to navigate to the proper place in the esophagus for treatment.

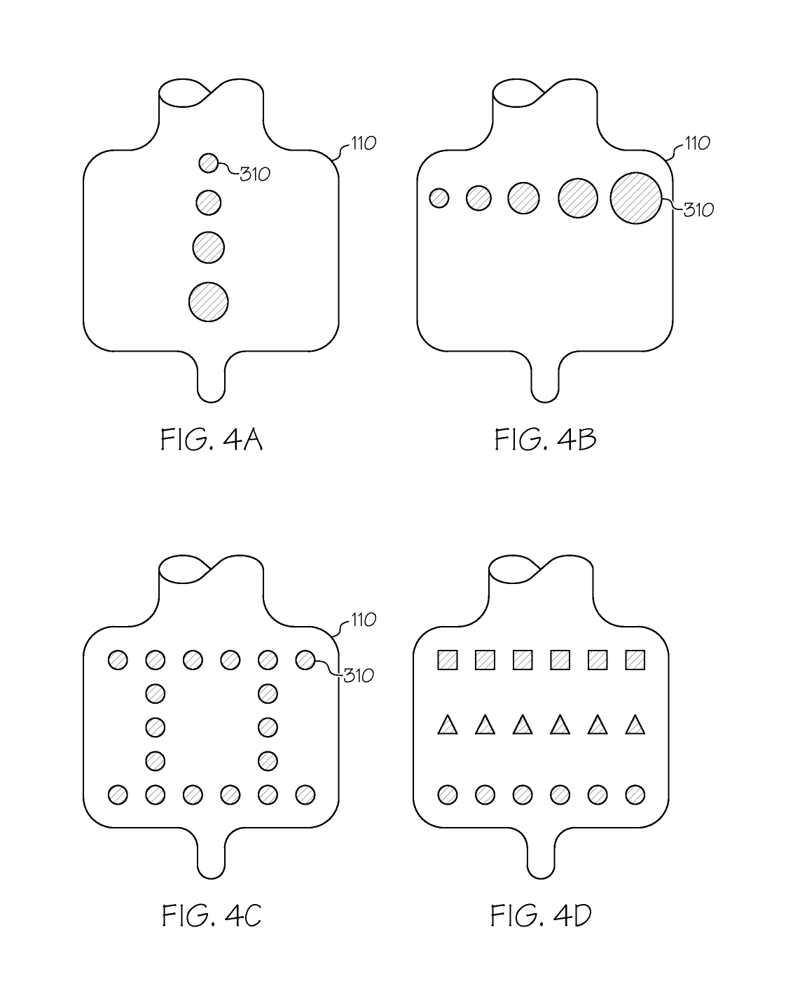

The shaped pieces 310 can be fabricated into sizes that can range from 1 to 50 microns depending upon the specific embodiment. The shaped pieces 310 are adhered to various parts of the exterior surface 300 of the balloon 110 using an adhesive layer 306. In some embodiments, the balloon 110, is plasma treated prior to bonding the shaped pieces 310 to improve the adhesion of the shapes to the exterior of the balloon 110 with the adhesive. The adhesive layer 306 can be any FDA-approved adhesive, such as an FDA-approved cyanoacrylate adhesive. The shaped pieces 310 can be placed upon the exterior surface 300 of the balloon 110 in various patterns. The patterns or fiducial marks may be based upon a standard X-Y grid, as shown in FIG. 2, or based upon other patterns that provide advantageous navigating properties for the surgeon using the balloon 110 for OCT imaging. Based upon the placement of one or more of the shaped pieces 310, a fiducial pattern can be created. In some embodiments, the shaped pieces 310 can be of various sizes, ranging from 1 to 50 microns, and arranged in a specific pattern. For example, the pattern can be formed from shaped pieces 310 having a gradient in size along the length of the balloon 110, indicating a z-position, as shown in FIG. 4A, or the pattern can be formed from shaped pieces 310 having a gradient axially around the circumference of the measurement region of the balloon 110, as shown in FIG. 4B. In other embodiments, the shaped pieces 310 can all be of the same size, and may be of different dye or pigment color, as shown in FIG. 4C. In still other embodiments, the pattern can include shaped pieces 310 having various shapes, as shown in FIG. 4D. The placement patterns, infrared transparency, and visual dye color can be varied depending on the embodiment.

In other embodiments, the transferrable markings 200, 202, 204, and 206 are transferred from the exterior surface of the balloon 110 to the adjacent area for investigation using a photodynamic technique. For example, the marking material may be selected such that it is activated upon exposure to light. Upon activation, the marking material may be released from the exterior surface of the balloon 110 and adhere to the surface of the adjacent area for investigation. The light may be, for example, an IR-light source. In some embodiments, the IR-light source used for activation may be an IR imaging light that is part of the IR-optical imaging device 104. For example, the IR-light source of the IR-optical imaging device 104 may illuminate the balloon 110 from the inside of the balloon 110, causing the transferrable markings 200, 202, 204, and 206 to be transferred from the exterior surface of the balloon 110 to the adjacent area for investigation. In other embodiments, the light may be another light source, such as a light-diffusing fiber embedded in the OCT probe 100. In some embodiments, the marking material can be activated with a wavelength from about 200 nm to about 1.5 μm. As above, the marking material may be selected such that it is fully set in less than about 60 seconds in order to prevent smudging.

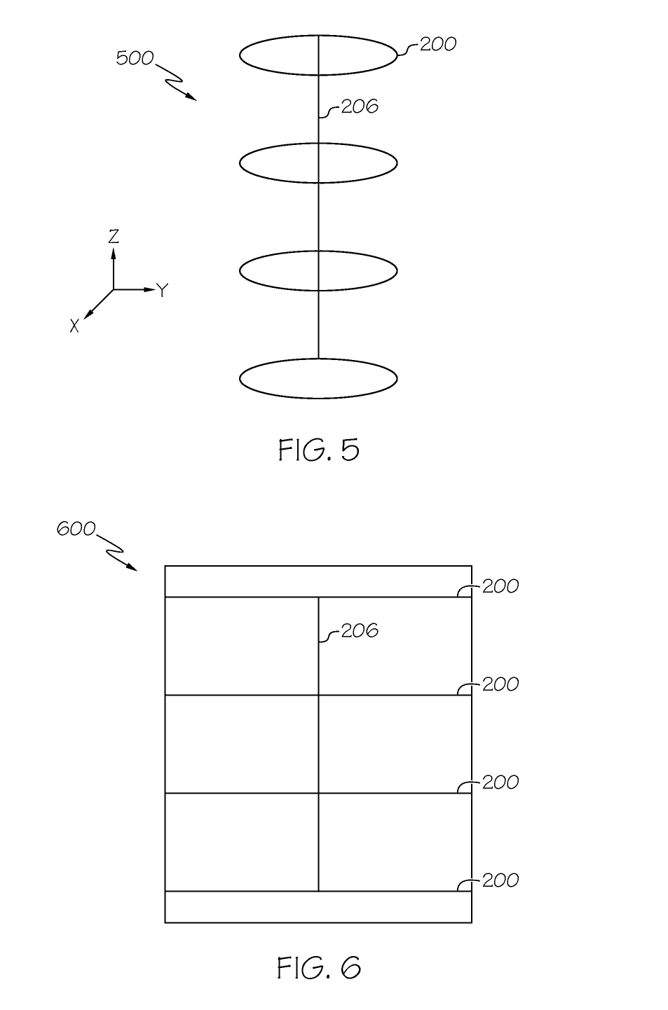

As described above, various embodiments include one or more transferrable markings formed from a partially IR-transmittable marking material, such as transferrable markings 200 and 206. FIG. 5 illustrates an exemplary embodiment 500 in which the transferrable markings 200 include four latitudinal markings parallel to the x-y plane and a longitudinal transferrable marking 206 parallel to the z-axis. Each of the markings shown in FIG. 5 is made using a partially IR-transmittable marking material. Accordingly, FIG. 5 illustrates the transferrable markings 200 and 206 on the area for investigation that are sensed by the IR-optical imaging device 104. In particular, the IR-optical imaging device 104 employs optical coherence tomography to capture data that can be used to generate a three-dimensional image, or tomogram, of the surrounding area for investigation (e.g., the esophagus). The transferrable markings 200 and 206 are sensed by the IR-optical imaging device 104 during the capture of the tomogram. The IR-optical imaging device 104 transmits the data corresponding to an image that includes the sensed transferrable markings 200 and 206 and information to generate the tomogram of the area for investigation to the imaging system.

The imaging system displays the data received from the IR-optical imaging device 104 as an image on a display device, such as a monitor, tablet, or other suitable display device. In various embodiments, the image can be displayed in a flat view 600, as shown in FIG. 6. As shown in FIG. 6, the transferrable markings 200 and 206 are present in the image. Any transferrable markings including IR-transmittable marking material, such as transferrable markings 202 and 204, are not shown in the displayed image. When displayed in the flat view 600, the transferrable markings 200 and 206 may form a grid, enabling a physician to form a surgical plan using the intersections of the transferrable markings 200 and 206 as reference points.

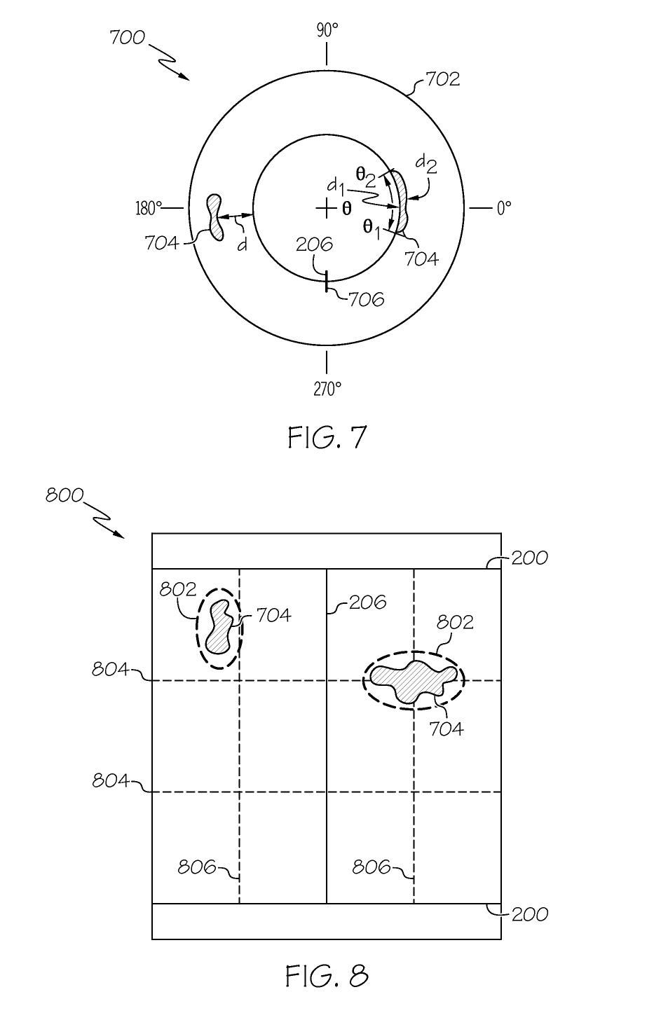

Turning now to FIG. 7, a slice of an image 700 is depicted. As used herein, the term “slice” refers to a cross-section of the esophagus 702 in the x-y plane, where z is coming out of the page. In practice, the imaging system can produce a plurality of slices, each corresponding to various portions of the area for investigation. For example, the imaging system can produce a slice for approximately every 0.015 mm along the length of the esophagus. Other slice thicknesses may be selected, depending on the particular embodiment and the features of the system. For example, the slice thickness may depend on an amount of system processing or memory available, the desired sensitivity, or other factors. The slice of the image 700 shown in FIG. 7 illustrates an esophagus 702 having two tissue anomalies 704. The displayed slice of the image 700 can further include reference markings, including an angular marking (θ), which are generated by the imaging system to provide a frame of reference. Accordingly, each of the tissue anomalies 704 can be identified on the slice of the image 700 by an angular θ range (θ₁-θ₂) and a depth d range (d₁-d₂). These location identifications, coupled with the location of the slice along the length of the esophagus, form the basis for the physician's surgical plan. For example, the locations identified as tissue anomalies 704 can be stored in a database, including the angular and depth ranges for each anomaly. Although the latitudinal transferrable markings 200 are not shown in the slice of the image 700 depicted in FIG. 7, the longitudinal transferrable marking 206 is visible as a shadow 706 into the depth d of the esophagus 702, enabling the physician to orient the slice of the image 700 within the area for investigation.

In addition to a plurality of slices along the length of the esophagus, the imaging system can also produce a plurality of depth profiles for display. As shown in FIG. 8, each depth profile 800 corresponds to a depth of the area for investigation. The depth profile 800 of FIG. 8 illustrates two tissue anomalies 704, along with the transferrable markings 200 and 206. The physician may use one or more of the depth profiles 800, either alone or in combination with other views, to formulate the surgical plan. For example, upon viewing the tissue anomalies 704, the physician can identify an area for excision 802. In various embodiments, the area for excision 802 includes a margin around the tissue anomaly 704. In some embodiments, the depth ranges obtained from the slice of the image 700, may enable the physician to select a plurality of depth profiles 800 to be viewed. For example, if the tissue anomalies 704 are both present from d₁-d₂, the physician may not need to view the depth profile 800 corresponding to d₃. However, in some embodiments, the physician may view additional depth profiles 800 to confirm the information represented by the slice of the image 700.

As shown in FIG. 8, the imaging system may superimpose additional markings, such as horizontal markings 804 and vertical markings 806, to enable the physician to better define the surgical plan. In various embodiments, the horizontal markings 804 correspond to transferrable markings 202 that include IR-transmittable marking material and the vertical markings 806 correspond to transferrable markings 204 that include IR-transmittable marking material. Accordingly, although the transferrable markings 202 and 204 are not sensed by the IR-optical imaging device 104 and do not form part of the image, the transferrable markings 202 and 204 are represented in the image by the horizontal markings 804 and the vertical markings 806. This virtual representation can enable a finer grid to be transferred to the area for investigation and viewed on the display device. In contrast, because the partially IR-transmittable marking material appears as a shadow on the image, transferring a fine grid with partially IR-transmittable marking material could result in an image in which the shadows obscure the image of the tissue, thereby rendering the image unusable for detection of tissue anomalies. The use of a fine grid can enable the physician to more specifically identify the area for excision 802.

As described above, the dimensions of the grid can vary depending on the particular embodiment, and the imaging system may include settings to enable the physician to adjust the dimensions of the superimposed additional markings to correspond to varying grid dimensions. For example, the physician may select a setting to superimpose additional markings that form a grid having 1 cm by 1 cm squares when a balloon catheter including transferrable markings 202 and 204 that form a grid having 1 cm by 1 cm squares is used. As another example, the physician may select a setting to superimpose additional markings that form a grid having 1 mm by 1 mm squares when a balloon catheter including transferrable markings 202 and 204 that form a grid having 1 mm by 1 mm squares is used.

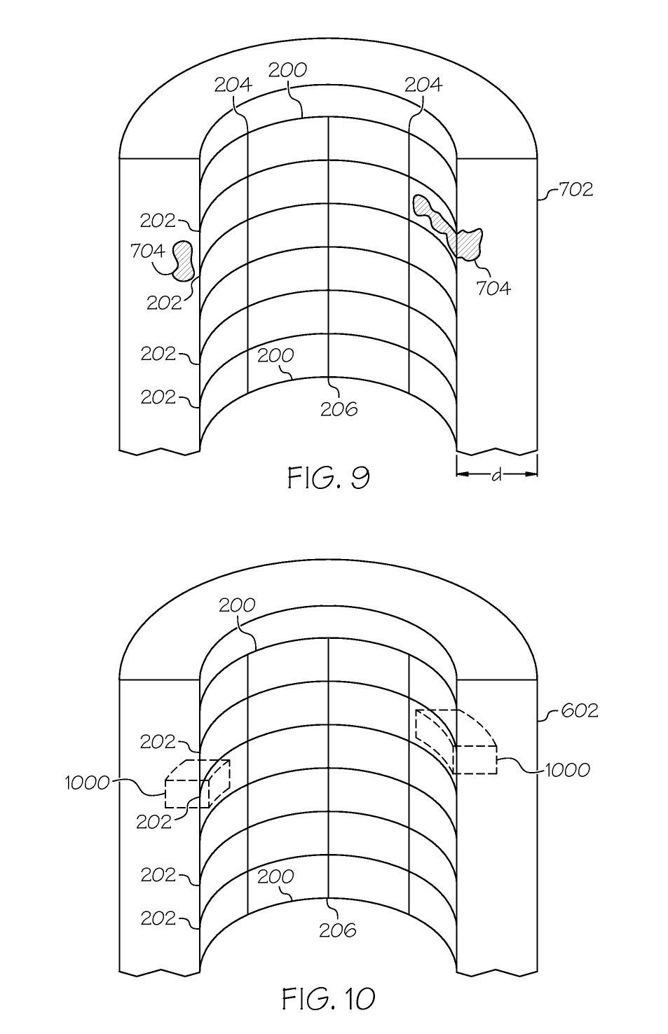

Once the physician has identified the areas for excision 802 using the slices of the image 700 and the depth profiles 800, in various embodiments, the physician can proceed to excise the tissue anomalies 704. FIG. 9 schematically depicts a pre-operative cross-section of an area for investigation according to one or more embodiments. As shown in FIG. 9, the esophagus 702 has a depth d, and includes two tissue anomalies 704. The esophagus 702 further includes a plurality of transferrable markings 200, 202, 204, and 206. As described above, a first portion of the transferrable markings, e.g., transferrable markings 200 and 206, include a partially IR-transmittable marking material and a second portion of the transferrable markings, e.g., transferrable markings 202 and 204, include an IR-transmittable marking material. Both the first portion of the transferrable markings and the second portion of the transferrable markings are visible under white light. Accordingly, FIG. 9 represents a view available to the physician via the endoscope inserted in the esophagus.

In order to excise the tissue anomalies 704, the physician registers the transferrable markings 200, 202, 204, and 206 on the area for investigation with the corresponding markings in the image displayed by the imaging system. In particular, the physician may match up the transferrable markings 202 and 204 on the surface of the esophagus 702 with the superimposed additional markings 804 and 806 on the diagnostic image, respectively. By registering the image with the actual markings on the surface of the esophagus 702, the physician can position the endoscopic tool adjacent to the identified location for each sample (e.g., the identified areas for excision 802). Each sample identified in the surgical plan can be excised until the surgical plan is completed.

FIG. 10 schematically depicts a post-operative cross-section of the area for investigation shown in FIG. 9. In particular, FIG. 10 illustrates the esophagus 702 including areas 1000 from which the tissue anomalies 704 were removed. FIG. 10 further includes the transferrable markings 200, 202, 204, and 206. In various embodiments, the transferrable markings 200, 202, 204, and 206 include a marking material that is formulated to fade or otherwise be released from the surface of the esophagus 702 over time. In some embodiments, the marking material is selected such that the transferrable markings 200, 202, 204, and 206 remain for a period of time to enable the area for investigation to be monitored. For example, the physician can excise a portion of the tissue anomalies 704 in order to biopsy the tissue anomalies 704. When the results of the biopsy indicate that the tissue anomaly 704 should be removed, the physician can return to the precise area for investigation, again registering the image with the transferrable markings 200, 202, 204, and 206 on the surface of the esophagus 702 with the corresponding markings in the image (including the superimposed additional markings 804 and 806), and remove the tissue anomaly 704.

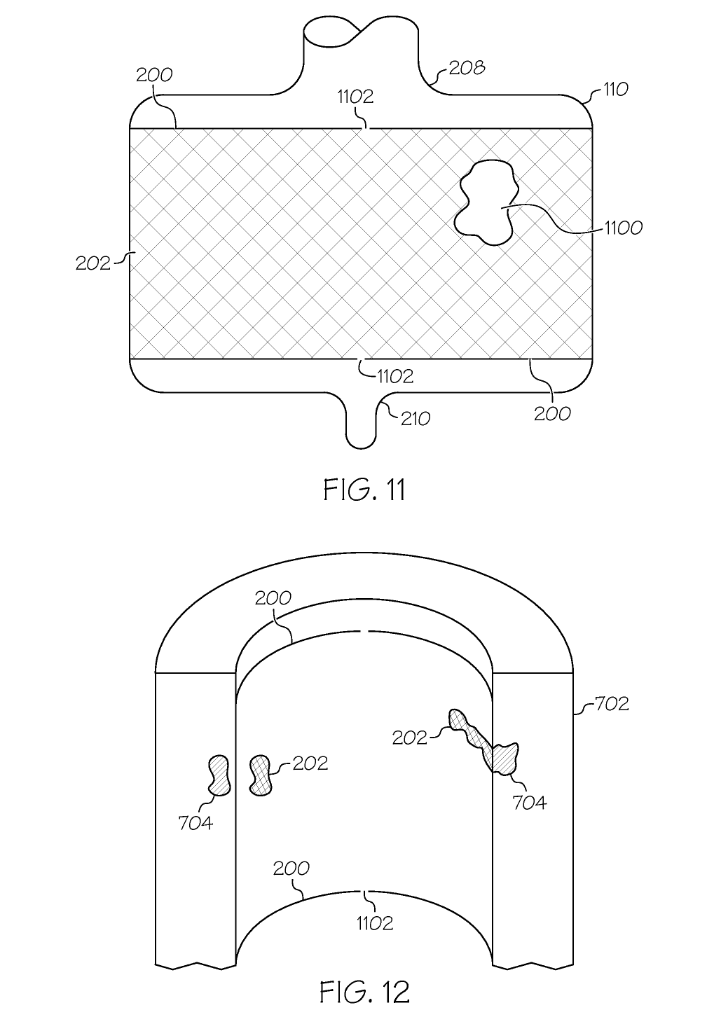

Although various embodiments described hereinabove include transferring markings having a known geometry to the area for investigation, in some embodiments, markings are selectively transferred from the exterior surface of the balloon to the area for investigation. As shown in FIG. 11, the exterior surface of the balloon 110 can include the transferrable marking 200 including a partially IR-transmittable marking material. Although shown in FIG. 11 as a horizontal line located near the proximal end 208 of the balloon 110 and a horizontal line located near the distal end 210 of the balloon 110, the transferrable marking 200 can be one or more spots, lines, or other markings sufficient to provide a key for registration of an image to the area for investigation.

In the embodiment shown in FIG. 11, the transferrable marking 202 covers a portion of the exterior surface of the balloon 110 between the horizontal line located near the proximal end of the balloon 110 and the horizontal line located near the distal end of the balloon 110 that make up the transferrable markings 200. In such embodiments, a photodynamic technique can be employed to transfer at least a portion of the transferrable marking 202 to the adjacent area for investigation. For example, when a tissue anomaly is visually identified on the surface of the adjacent area for investigation using the IR-optical imaging device 104, the area 1100 can be activated with light such that the marking material in the area 1100 is transferred to the adjacent area for investigation. In such embodiments, when the physician removes the OCT probe, the endoscopic tool for excision can be positioned adjacent to the tissue anomaly 704 by identifying the transferrable marking 202 on the surface of the esophagus 702, as shown in FIG. 12. The physician can excise the area covered with the transferrable marking 202 in the esophagus 702. Accordingly, the marking material is removed with the tissue anomaly 704, leaving behind less foreign substance in the patient's body.

In the embodiment shown in FIGS. 11 and 12, the balloon 110 also includes a longitudinal marking 1102. The longitudinal marking 1102 serves to orient the transmitted image with respect to the cylinder, similar to the longitudinal transferrable marking 206. However, the longitudinal marking 1102 is defined by a space where the marking material of the transferrable marking 200 is not transferred to the adjacent area for investigation. This is in contrast to the forming of the longitudinal transferrable marking 206 by transferring a partially IR-transmittable marking material to the adjacent area for investigation.

In various embodiments, the longitudinal markings, including longitudinal transferrable marking 206 and the longitudinal marking 1102, can enable the imaging device to adjust the image displayed by the display device. For example, the imaging device can use the longitudinal markings to correct a skew or rotation of the image.

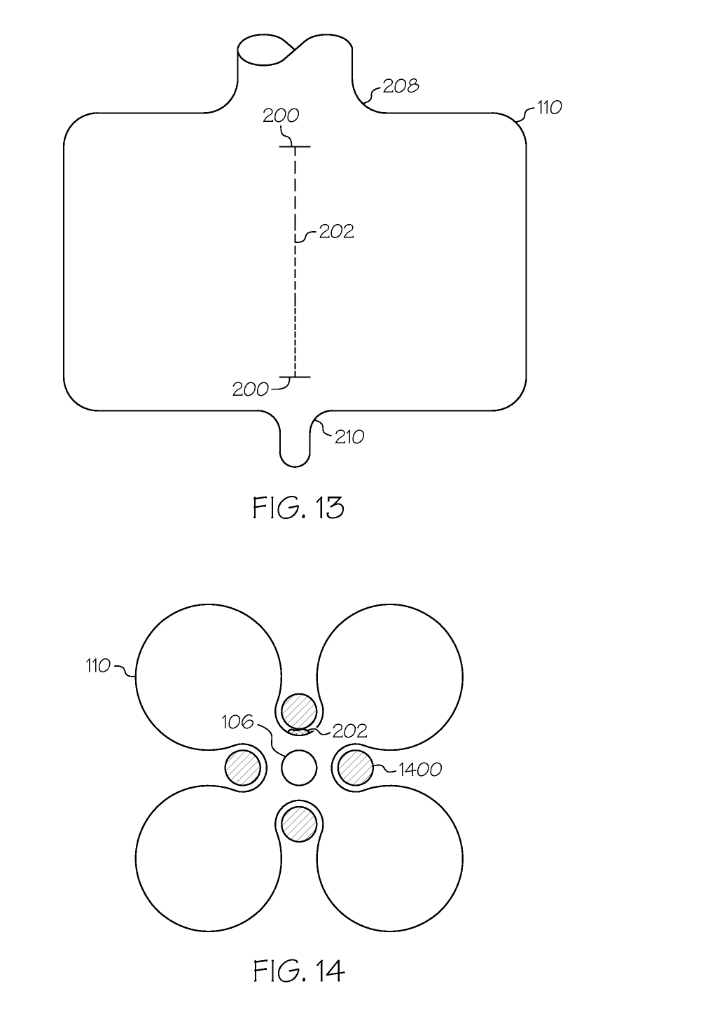

FIG. 13 depicts yet another embodiment having a transferrable marking 200 formed from a partially IR-transmittable marking material and a transferrable marking 202 formed from IR-transmittable marking material. In the embodiment shown in FIG. 13, the transferrable marking 200 does not extend around the complete circumference of the balloon 110. Additionally, the transferrable marking 202 includes a pattern that varies from the proximal end 208 of the balloon 110 to the distal end 210 of the balloon 110. This type of longitudinal marking can provide the visual identification information while reducing any potential adverse effect from packing the collapsed balloon for transport through the esophagus. For example, when the balloon 110 is collapsed and folded for transport through the endoscope into the esophagus, the marking material making up the transferrable markings may become separated from the exterior surface of the balloon 110, which can result in skewed or even untransferred markings. By reducing the width of the transferrable markings along the circumference of the balloon 110, the balloon 110 may be folded in a manner to more fully protect the transferrable markings.

FIG. 14 shows one example of folding the balloon 110. In the embodiment shown in FIG. 14, the balloon 110 is folded such that the transferrable marking 202 is folded in towards the inner lumen 106. The transferrable marking 202 can be folded in towards the inner lumen 106 by pressing a rod 1400 against the transferrable marking 202, directing the surface of the balloon 110 towards the inner lumen 106. In various embodiments, more than one rod 1400 can be used to collapse the balloon 110. Once folded, the proximal end of the balloon 110 can be twisted relative to the distal end of the balloon 110, and the rods 1400 can be removed. Other methods of packing and/or folding the balloon 110 can be employed depending on the particular embodiment.

In use, the surgeon inserts the pre-folded balloon 110 with the transferable markings 202 into the endoscope and positions the balloon in the esophagus. Fluid is added to the balloon 110 to inflate it and initiate contact of the exterior surface of the balloon the wall of the esophagus. The transferrable markings 202 are transferred from the exterior surface 300 of the balloon 110 to the wall of the esophagus. For example, the moist environmental conditions of the wall of the esophagus can moisten the water soluble dye or pigment infused polymer, causing the polymer to dissolve, and releasing the dye or pigments to be in contact with the esophagus during a diagnostic OCT scan. Once the OCT scan has been completed and analyzed, the balloon is deflated and removed from the patient's esophagus, leaving behind planned fiducial markings for the surgeon to be utilized in their later excision of diseased tissues.

### Examples

Various embodiments will be further clarified by the following example.

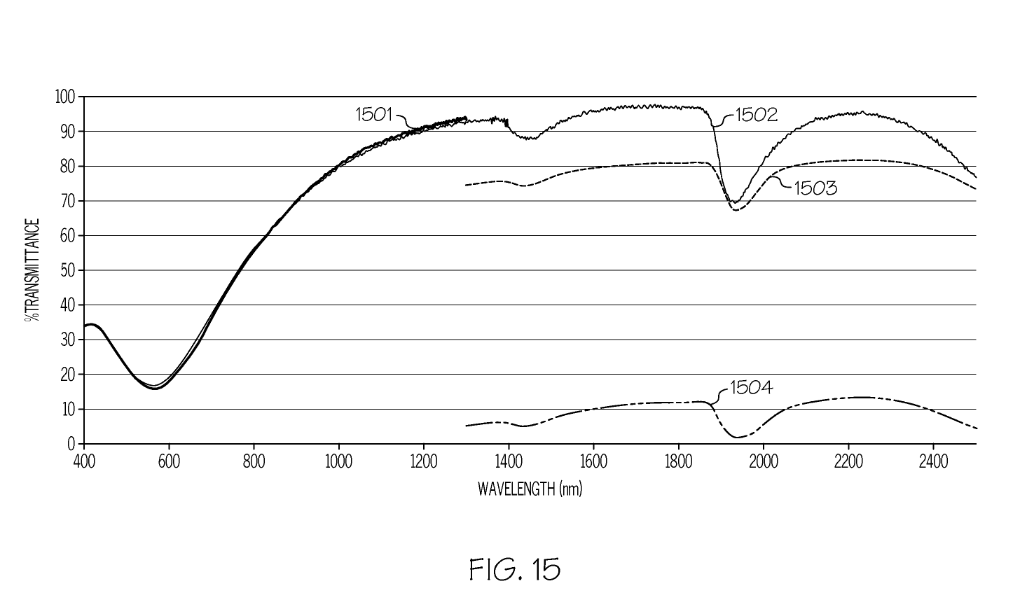

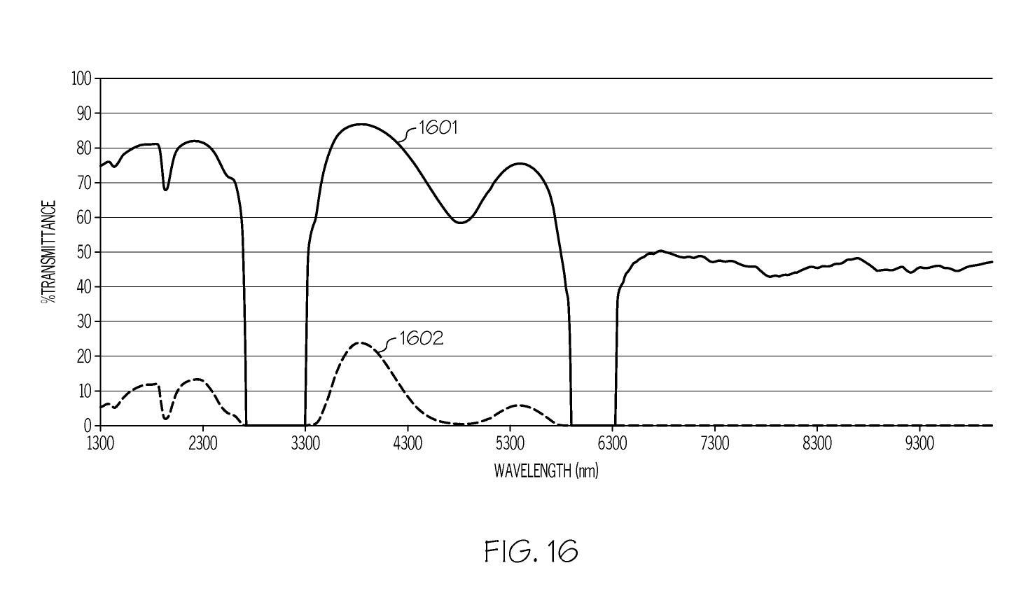

Transmittance properties of CARCO WS-860 black ink were measured according to various methods to determine its suitability for use in various embodiments. FIGS. 15 and 16 illustrate the results of an example in which transmittance was measured over the visible and IR spectra. In both FIGS. 15 and 16, the wavelength (in nm) is illustrated along the x-axis while the transmittance (in % transmittance) is illustrated along the y-axis.

The sample of CARCO WS-860 black ink was measured using two methods. According to one method, a 1 ml portion was diluted in 99 ml DI water to produce a 1 volume % solution. The transmittance of this sample was measured using a Perkin-Elmer 950 #2 spectrophotometer with 150 mm diameter sphere detector between wavelengths of 1300 nm and 400 nm using a 2 mm pathlength cuvette ratioed against the cuvette filled with DI water to yield a sample transmittance with reflection loss removed. Additionally, the following parameters were used: 3.0 nm spectral bandwidth PMT, InGaAs detector in servo slit mode, gain of 18, scan speed of 220 nm/min, scan step size of 2 nm, signal average time of 0.5 seconds, and an aperture of 10 mm diameter. The transmittance is represented by line 1501 in FIG. 15.

Additionally, an undiluted portion of sample was pressed between dry fused silica plates and measured using the above spectrophotometer parameters for 2500 nm-400 nm transmittance ratioed against a fused silica plate to yield a sample transmittance with reflection loss removed. Thickness measurement of the plates with and without sample yielded a sample thickness of 0.02 mm. The transmittance is represented by line 1502 in FIG. 15. A visual comparison of lines 1501 and 1502 confirms that the 1% solution at 2 mm pathlength is equivalent to a 0.02 mm solid sample pathlength. Both lines 1501 and 1502 illustrate the increased, but less than 100%, transmittance of the ink in the IR-range, indicating that the ink is visible in the visible spectrum (e.g., to the naked eye) as well as in the images produced according to one or more embodiments described hereinabove.

The IR transmittance was then measured using FTIR by pressing a portion of sample between KBr plates. Sample transmittance was ratioed against a KBr plate. Measurements were made using a Nicolet 670 FTIR using the following parameters: resolution of 8 cm⁻¹; 128 scans; gain of 1; iris aperture opening of 30%; 10 mm diameter aperture. The transmittance of the material (e.g., adjusted to remove the transmittance attributable to the KBr plates) is represented by line 1504 in FIG. 15. Sample thickness was determined by subtracting plate thickness from the total thickness of sample between plates. Sample thickness was determined to be 0.2 mm pathlength. Accordingly, when normalized to 0.02 mm, line 1503 in FIG. 15 can be compared to line 1502. A visual comparison of lines 1502 and 1503 indicates that the sample measured using FTIR exhibits lower transmittance than the sample measured between fused silica plates. However, this difference may be attributed to the FTIR measurement being more prone to scatter losses due to the sample being further from the detector as well as the increased scattering of the KBr plates as compared to the fused silica plates.

FIG. 16 illustrates the transmittance of a 0.2 mm sample of CARCO WS-860 in the far-IR range. The parameters for the FTIR measurement remained the same as in the previous measurement, with the exception of the wavelength range over which the measurement was taken. In FIG. 16, line 1601 illustrates the transmittance of the sample independent of the KBr plates normalized to 0.02 mm while line 1602 represents the transmittance of the sample independent of the KBr plates as measured by the FTIR for a sample having a thickness (as calculated by subtracting plate thickness from the total thickness of sample between plates) of 0.2 mm.

The data illustrates the suitability of the ink for use in accordance with one or more embodiments described herein. In particular, CARCO WS-860 ink is observable in the visible spectrum and further exhibits an amount of transmittance in the IR range (greater than about 5% but less than about 95%) such that it can be detected when applied to a balloon and imaged using IR-imaging techniques as described herein.

It should now be understood that embodiments of the present disclosure enable registration of IR-optical images with an area for investigation by transferring a plurality of markings from an exterior surface of a balloon to the adjacent area for investigation. The plurality of markings includes at least one marking of a partially IR-transmittable marking material and at least one marking of an IR-transmittable marking material. Various embodiments enable the image to be registered using the markings of partially IR-transmittable marking material, while the markings of IR-transmittable marking material enable specific location identification for areas to be excised. In particular, the combination of partially IR-transmittable and IR-transmittable markings enable a fine grid or other marking to be transferred to the esophageal tissue (or other tissue) without obscuring the image and correlated to an image displayed by the imaging system. Superimposed markings on the displayed image which correspond to the IR-transmittable markings enable a physician to specifically identify one or more locations for excision, and to register those locations with the IR-transmittable markings on the esophageal tissue using the partially IR-transmittable markings.

In a first aspect, the disclosure provides a balloon catheter having a balloon located at a distal end of the balloon catheter, the balloon including a first transferrable marking comprising a partially IR-transmittable marking material on an exterior surface of the balloon, and a second transferrable marking comprising an IR-transmittable marking material on the exterior surface of the balloon.

In a second aspect, the disclosure provides the balloon catheter of the first aspect, further including: an inner lumen positioned within an outer lumen, the inner lumen extending through the balloon; and a fluid passageway between the inner lumen and the outer lumen, the fluid passageway transferring a fluid to and from the balloon to inflate and deflate the balloon.

In a third aspect, the disclosure provides the balloon catheter of the first or second aspect, further including an optical imaging device positioned within the inner lumen of the balloon catheter, the optical imaging device capturing one or more images through the inner lumen and the balloon.

In a fourth aspect, the disclosure provides the balloon catheter of any of the first through third aspects, wherein the optical imaging device comprises an IR-optical imaging device.

In a fifth aspect, the disclosure provides the balloon catheter of any of the first through fourth aspects, wherein the balloon further comprises a time-release coating over the first transferrable marking and the second transferrable marking.

In a sixth aspect, the disclosure provides the balloon catheter of any of the first through fifth aspects, further including a silicone release liner comprising a release surface and a non-release surface, wherein the first transferrable marking and the second transferrable marking are disposed on the release surface of the silicone release liner; and wherein the silicone release liner is affixed to the exterior surface of the balloon such that the non-release surface of the silicone release liner is positioned adjacent to the exterior surface of the balloon.

In a seventh aspect, the disclosure provides the balloon catheter of any of the first through sixth aspects, wherein at least one of the first transferrable marking and the second transferrable marking comprises a dye and a PEG sealant.

In an eighth aspect, the disclosure provides an optical coherence tomography (OCT) probe including an IR-optical imaging device for communication with an imaging system; and a balloon catheter having a balloon at a distal end of the balloon catheter, the balloon having a plurality of transferrable markings on an exterior surface of the balloon. The first portion of the plurality of transferrable markings includes a partially IR-transmittable marking material and a second portion of the plurality of transferrable markings includes an IR-transmittable marking material. The IR-optical imaging device senses the first portion of the plurality of markings and does not sense the second portion of the plurality of markings.

In a ninth aspect, the disclosure provides any of the first through eighth aspects wherein the IR-optical imaging device captures an image through the balloon at the distal end of the balloon catheter.

In a tenth aspect, the disclosure provides any of the first through ninth aspects, wherein the IR-optical imaging device includes an IR-light source.

In an eleventh aspect, the disclosure provides any of the first through tenth aspects, wherein the IR-light source causes the plurality of transferrable markings to be transferred from the exterior surface of the balloon to an adjacent area for investigation.

In a twelfth aspect, the disclosure provides any of the first through eleventh aspects, the balloon further including a coating over the plurality of transferrable markings.

In a thirteenth aspect, the disclosure provides any of the first through twelfth aspects, wherein the coating is a time-release coating.

In a fourteenth aspect, the disclosure provides a method for performing optical coherence tomography (OCT) may include positioning an OCT imaging device adjacent to an area for investigation; transferring a plurality of markings from the OCT imaging device to the adjacent area for investigation, a first portion of the plurality of markings comprising a partially IR-transmittable marking material and a second portion of the plurality of markings comprising an IR-transmittable marking material; and transmitting an image from the optical imaging device, the image comprising at least the first portion of the plurality of markings and a three-dimensional model of the area for investigation.

In a fifteenth aspect, the disclosure provides the method of the fourteenth aspect, further including superimposing additional markings on the transmitted image, the additional markings corresponding to the second portion of the plurality of markings; identifying, using the transmitted image and superimposed additional markings, a location of a sample to be removed from the area for investigation; and positioning an endoscopic tool adjacent to the identified location of the sample by registering the second portion of the plurality of markings on the area for investigation that correspond to the superimposed additional markings on the image.

In a sixteenth aspect, the disclosure provides the method of the fourteenth or fifteenth aspects, wherein the plurality of markings are visible in white light.

In a seventeenth aspect, the disclosure provides the method of any of the fourteenth through sixteenth aspects, wherein the first portion of the plurality of markings includes at least one longitudinal marking.

In an eighteenth aspect, the disclosure provides the method of any of the fourteenth through seventeenth aspects, wherein the first portion of the plurality of markings forms a grid.

In a nineteenth aspect, the disclosure provides the method of any of the fourteenth through eighteenth aspects, wherein the OCT device includes a balloon located at a distal end of the OCT device, and wherein the plurality of markings are transferred from an exterior surface of the balloon to the adjacent area for investigation.

In a twentieth aspect, the disclosure provides the method of any of the fourteenth through nineteenth aspects, wherein transferring the plurality of markings from the OCT imaging device includes illuminating a balloon having the plurality of markings on an exterior surface with a light.

It will be apparent to those skilled in the art that various modifications and variations can be made to the embodiments described herein without departing from the spirit and scope of the claimed subject matter. Thus it is intended that the specification cover the modifications and variations of the various embodiments described herein provided such modification and variations come within the scope of the appended claims and their equivalents.

1. A balloon catheter comprising:
a balloon located at a distal end of the balloon catheter, the balloon comprising:
a first transferrable marking comprising a partially IR-transmittable marking material on an exterior surface of the balloon; and
a second transferrable marking comprising an IR-transmittable marking material on the exterior surface of the balloon.

2. The balloon catheter of claim 1, further comprising:
an inner lumen positioned within an outer lumen, the inner lumen extending through the balloon; and
a fluid passageway between the inner lumen and the outer lumen, the fluid passageway transferring a fluid to and from the balloon to inflate and deflate the balloon.

3. The balloon catheter of claim 2, further comprising:
an optical imaging device positioned within the inner lumen of the balloon catheter, the optical imaging device capturing one or more images through the inner lumen and the balloon.

4. The balloon catheter of claim 3, wherein the optical imaging device comprises an IR-optical imaging device.

5. The balloon catheter of claim 1, wherein the balloon further comprises a time-release coating over the first transferrable marking and the second transferrable marking.

6. The balloon catheter of claim 1, further comprising:
a silicone release liner comprising a release surface and a non-release surface, wherein the first transferrable marking and the second transferrable marking arc disposed on the release surface of the silicone release liner; and
wherein the silicone release liner is affixed to the exterior surface of the balloon such that the non-release surface of the silicone release liner is positioned adjacent to the exterior surface of the balloon.

7. The balloon catheter of claim 1, wherein at least one of the first transferrable marking and the second transferrable marking comprises a dye and a PEG sealant.

8. An optical coherence tomography (OCT) probe comprising:
an IR-optical imaging device for communication with an imaging system; and
a balloon catheter having a balloon at a distal end of the balloon catheter, the balloon comprising a plurality of transferrable markings on an exterior surface of the balloon,
wherein a first portion of the plurality of transferrable markings comprises a partially IR-transmittable marking material and a second portion of the plurality of transferrable markings comprises an IR-transmittable marking material, wherein the IR-optical imaging device senses the first portion of the plurality of markings and does not sense the second portion of the plurality of markings.

9. The OCT probe of claim 8, wherein the IR-optical imaging device captures an image through the balloon at the distal end of the balloon catheter.

10. The OCT probe of claim 8, wherein the IR-optical imaging device comprises an IR-light source.

11. The OCT probe of claim 10, wherein the IR-light source causes the plurality of transferrable markings to be transferred from the exterior surface of the balloon to an adjacent area for investigation.

12. The OCT probe of claim 8 the balloon further comprising a coating over the plurality of transferrable markings.

13. The OCT probe of claim 12, wherein the coating is a time-release coating.

14. A method for performing optical coherence tomography (OCT) comprising:
positioning an OCT imaging device adjacent to an area for investigation;
transferring a plurality of markings from the OCT imaging device to the adjacent area for investigation, a first portion of the plurality of markings comprising a partially IR-transmittable marking material and a second portion of the plurality of markings comprising an IR-transmittable marking material; and
transmitting an image from the optical imaging device, the image comprising at least the first portion of the plurality of markings and a three-dimensional model of the area for investigation.

15. The method of claim 14, further comprising:
superimposing additional markings on the transmitted image, the additional markings corresponding to the second portion of the plurality of markings;
identifying, using the transmitted image and superimposed additional markings, a location of a sample to be removed from the area for investigation; and
positioning an endoscopic tool adjacent to the identified location of the sample by registering the second portion of the plurality of markings on the area for investigation that correspond to the superimposed additional markings on the image.

16. The method of claim 14, wherein the plurality of markings are visible in white light.

17. The method of claim 14, wherein the first portion of the plurality of markings comprises at least one longitudinal marking.

18. The method of claim 14, wherein the first portion of the plurality of markings comprises a grid.

19. The method of claim 14, wherein the OCT device comprises a balloon located at a distal end of the OCT device, and wherein the plurality of markings are transferred from an exterior surface of the balloon to the adjacent area for investigation.

20. The method of claim 14, wherein transferring the plurality of markings from the OCT imaging device comprises illuminating a balloon having the plurality of markings on an exterior surface with a light.
